(19) United States
(12) Patent Application Publication
(10) Pub. No.: US 2011/0212895 A1
(43) Pub. Date: Sep. 1, 2011

(54) Treatment of Cognitive and Learning Impairment

(75) Inventors: Giovanni Diana, Rome (IT); Carla Fiorentini, Rome (IT)

(73) Assignee: Instituto Superiore Di Sanita

(21) Appl. No.: 11/887,756

(22) PCT Filed: Apr. 4, 2006
(86) PCT No.: PCT/EP2006/003811

§ 371 Date: Feb. 7, 2008

(30) Foreign Application Priority Data
Apr. 4, 2005 (GB) 0506797.0

(51) Int. Cl. A61P 25/18; C12N 9/14; A61P 25/24; A61P 25/16; A61P 25/28; A61P 25/00; A61K 38/16; A61K 31/7088; C07K 14/00; C07K 16/40; A61P 3/00

(52) U.S. Cl. 514/17.5; 435/195; 514/17.6; 514/17.7; 514/17.8; 514/17.9; 514/21.2; 514/44 R; 530/350; 530/387.3

(57) ABSTRACT

Constitutive activators of Rho GTPases are useful in treating learning an cognitive disorders.

The present invention relates to the use of known proteins in the treatment of cognitive and learning disorders.

Improving learning and treating cognitive deficiencies have been research targets for decades, if not centuries. However, as yet, the biological and chemical processes that take place during learning and cognitive processes remain shrouded in mystery, with many possible avenues remaining to be explored.

Learning is thought to be associated with the re-arrangement of synaptic connections in the Central Nervous System (CNS). Changes have been detected in the protrusions of dendritic trees (“dendritic spines”), after intensive learning training [17,28]. In addition, Long-Term Potentiation (LTP), a phenomenon which models the activity-dependent changes of synaptic efficacy believed to represent the cellular basis of learning, is associated with morphological changes in dendritic spines [16, 21, 26].

There is experimental evidence that the neuronal actin cytoskeleton is involved in dendritic spine morphology and re-arrangement [10, 12, 35]. In the last few years, it has been shown that GTPases belonging to the Rho family, a class of hydrolase that is highly conserved during evolution [13], play a pivotal role in the regulation of actin assembly and polymerisation and actomyosin contraction [8, 10, 14, 20, 30], thus controlling the dynamics of neuron morphology [18, 19, 22, 32, 33]. The Rho GTPase family includes RhoA, Rac1, and CDC42, and controls actin dynamics, a mechanism capable of regulating dendritic spine morphology.

In mental retardation, both spine morphology and Rho GTPase signalling are consistently implicated [2, 4, 15, 25]. There has been speculation that genetic polymorphism of Rho GTPases might underlie differences in cognition abilities among healthy subjects [27]. In addition, hippocampal CA1 neurotransmission, which is associated with activation of Rho GTPases [23], can be modified through drugs affecting this protein family [24]. However, in spite of all of the evidence about the potential role of Rho-GTPase in the structural plasticity of the CNS, there is no evidence that their selective activation leads to increased learning abilities and memory.

One reason for this is that there are few available molecules that can selectively modulate the activity of Rho-GTPases. Indeed, genes involved in Mental Retardation and their products have been proposed for activating cerebral Rho-GTPases and, possibly, enhancing learning ([36], Endris et al, PNAS Sep. 3, 2002, Vol 99 number 19 pp. 117754-11759). On the contrary, it has also been shown that activating Rho-GTPases, such as RhoA, affects spine morphology and models some feature of a well known form of mental retardation ([37], Govek et al Nature Neuroscience 7, number 4, April 2004 p 364). However, whatever the effects of activating Rho-GTPases, these human Rho-modulating proteins and genes play a strictly intracellular role, and do not possess the intrinsic ability of activating Rho-GTPases when administered in the extracellular space. Hence, their therapeutic use presents some major technical problems.

Cytotoxic Necrotising Factor 1 (CNF1), a 114 kDa protein toxin which determines the potential pathogenic activity of Escherichia coli, induces a sustained activation of RhoA, Rac1 and CDC42 in intact cells [34]. In other words, the toxin possess a molecular machinery which allows for the enzymatic domain to enter the cell. The enzymatic activity, which is shared by Escherichia coli CNF2 and Bordetella Dermonecrotic Toxin (DNT), is accomplished constitutively, through site specific deamidation of a gln residue to glu, and requires the presence of cysteine in position 866 (activity is lost if cysteine is substituted for serine at this position), as well as histidine in position 881 [5,29]. The sustained effect of this molecule and those belonging to its class represent another property that can make convenient their use in human therapy. In fact, the molecules might be administered rather infrequently and still retain a continuous therapeutic effect.

Surprisingly, we have now established that a select group of microbial toxins, which specifically targets Rho GTPases, can have a stimulatory effect on the learning and cognitive processes.

Thus, in a first aspect, the present invention provides the use of a Rho GTPase activator in the manufacture of a medicament for the treatment of learning and cognitive disorders, wherein the Rho GTPase activator is selected from CNF1, CNF2, DNT or a mutant or variant thereof, provided that the Rho GTPase activator is effective either to deamidate or to transglutaminate Gln63 in RhoA and/or Gln61 in Rac1 and/or Gln61 in CDC42.

As noted above, CNF1 and CNF2 are cytotoxic necrotising factors expressed by certain pathogenic strains of E. coli, while DNT is a related dermonecrotic toxin expressed by various Bordetella spp. It will be appreciated that the present invention extends to other family members of the Rho GTPase activators, in particular bacterial toxins capable of acting as Rho GTPase activators, together with their mutants and variants. For example, another family member is expressed by Yersinia.

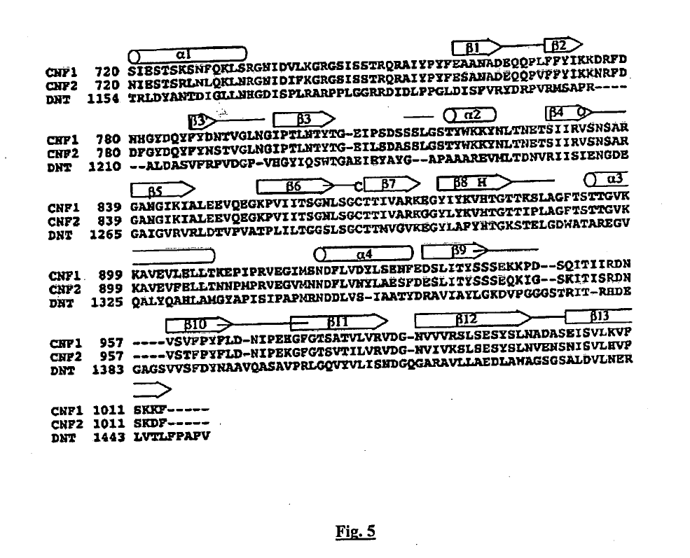

In general, the family is characterised by a highly conserved active pocket. The sequence for this pocket for CNF1, CNF2 and DNT is shown in accompanying FIG. 5, from which it can be seen that there is a highly conserved sequence of about 280 amino acids, of which residues 728 to 893, relative to the CNF sequences, or corresponding thereto, show a particularly high degree of homology, as previously shown [5].

The active pocket of the bacterial toxin Rho GTPase activators is capable of both deamidation and transglutamination, and the choice of whether to substitute with a primary amine or hydroxyl group depends largely on the prevailing conditions and the body of the molecule. For example, DNT has a preference towards transglutamination, rather than deamidation as demonstrated largely by CNF1 and CNF2.

Without being bound by theory, it is also believed that the body of the molecule has a significant bearing on the specificity of the active, or catalytic, site. Thus, in an alternative aspect, the present invention extends to a chimaeric molecule comprising a Rho GTPase activator, preferably a bacterial toxin Rho GTPase activator, active site and a further element, such as part or all of an antibody molecule, or a sequence containing a binding domain specific for selected target receptors in selected target cells. Such chimaeric molecules may also comprise a combination of a bacterial toxin Rho GTPase activator site and receptor binding subunits derived from other suitable proteins or binding molecules. It is particularly preferred that the further element is derived from a naturally occurring Rho-GTPase activator, or mutant or variant thereof, and it is more preferred that the whole activator, including active site, is derived from a naturally activator, especially CNF1.

Where reference is made to the active site of the Rho-GTPase activator, it is preferred that the active site is provided in the form of the whole catalytic domain of the activator. The catalytic domain of the activator comprises the active site and ensures that the 3-D spatial configuration, and thus enzymatic activity, is retained. The active sites and catalytic domains are discussed further below.

Rho-GTPases are known to be linked to Mental Retardation and, therefore treatment thereof, see for instance WO 03/030836, Govek et al Nature Neuroscience 7, number 4, April 2004 p 364), WO 03/095483 and Endris et al (PNAS Sep. 3, 2002, Vol 99 number 19 pp. 117754-11759). Furthermore, the bacterial toxins CNF1, CNF2 and DNT are known to be Rho-GTPase activators. For CNF1 and CNF2, see for instance Boquet (Annals New York Acad Sci, Vol 886, 41999, pp. 83-90). For DNT, see for instance Masuda et al (Infection & Immunity, Vol 70, No. 2, February 2002, pp. 998-1001), and Schmidt et al (Infection & Immunity, Vol 69, No. 12, December 2001, pp. 7663-7670).

However, the widely held view in the art, as shown by Govek et al, is that activation of Rho-GTPases lead to mental retardation. Indeed, the teaching of Govek et al merely confirms the view presented previously by Billuart et al, ([4], Nature 1998 Apr. 30; 392(6679):923-6). According to these authors, constitutive activation of cerebral Rho-GTPases in patients lacking Oligophrenin-1 affects cell migration and outgrowth of axons and dendrites in vivo, leading to mental retardation. Moreover, there is evidence that inhibiting RhoA and Rac1 increases the magnitude of LTP. LTP is a laboratory phenomenon modelling changes in synaptic efficacy thought to underlie learning ([24] O'Kane et al., 2004).

The present invention, therefore, runs contrary to the established teaching of the art. We have, surprisingly, shown that Rho-GTPase activators can, instead, be useful in treating learning and cognitive impairment, rather than leading to mental retardation. In other words, we have discovered that Rho-GTPase activators, especially bacterial toxins according to the present invention, reduce learning and cognitive impairment rather than increase it. Until now, their use has not been shown to improve learning in animal models.

As mentioned above, the bacterial toxins CNF1, CNF2 and DNT are well known in the art as Rho-GTPase activators. However, as discussed, activation by these bacterial toxins has never been shown in the CNS. Hence, the use of these toxins as Rho-GTPase activators is not known for treatment of learning disorders.

Whilst the use of some Rho-GTPase activating proteins of human origin and their encoding genes has been disclosed for treating mental retardation, their use is not actually feasible in humans at present. This is due to the technical difficulties in making them access the internal space of the cell and, ultimately, the target molecules.

However, one of the advantages of the bacterial toxins of the present invention is that the molecule can exerts its effects in “intact” neural cells. In other words, CNF1 and its related toxins possess a “binding” and an “internalization” domain that can carry their enzymatic effect into the neuron from the surrounding extracellular space. Altogether, this makes their use feasible. Moreover, these toxins permanently activate Rho-GTPases by modification, preferably deamidation, of a single amino acid. This property makes their effect sustained and allows for infrequent administration. Conversely, human Rho-GTPase activators only have a transient effect.

It will be appreciated that, given the number of different family members that is so far known, the body of the activator can be substantially varied, while the active site itself may also be varied. In particular, the body of the molecule is preferably based on an existing Rho GTPase activator bacterial toxin, and may be varied for a variety of reasons which may include, for example, variations resulting from genetic modifications useful in the preparation of the activator, and variations to the activator molecule to enhance formulation or to introduce desirable functionality. All such variations may be considered to be comprised within the term “variants”, as may naturally occurring variants and other family members. The term “mutants” generally relates to sequences modified either at the genetic or peptide level, and which are related to the naturally occurring molecule by such mutations as insertions, inversions, deletions and substitutions.

In particular, nucleic acid sequences encoding activators of the invention may be varied substantially, while still encoding the same protein, and advantage may be taken of this in order to enhance expression in a heterologous host. Other substitutions may be made within the peptide sequence itself and, even in the active site, substitutions may be made, especially at those locations shown in FIG. 5, where there is a lack of homology between sequences. FIG. 5 also demonstrates the possibility of deletions and/or insertions in certain areas.

Preferably, the mutant or variant shares at least 70%, more preferably at least 90%, more preferably at least 95%, more preferably at least 99%, and most preferably at least 99.9% homology with CNF1, CNF or DNT.

The peptide sequences for CNF1, CNF2, from E. coli, and DNT are provided in SEQ ID NOS. 1, 2 and 3, respectively. The catalytic domains are from amino acids 721-1013 in CNF1 and CNF2 and from 1167-1464 in DNT. The active sites are mentioned below. The Dermonecrotic toxin (DNT) sequence provided in SEQ ID NO. 3 is derived from Bordetella pertussis Tohama I and it will be appreciated that there is some variation between species.

In FIG. 5, the residues in the active site pocket that are conserved among the toxins were mutated to determine their contribution to activity. Asn 835 and Ser 864, which bracket Cys 866, were individually mutated to alanines. The activity of these mutants was assessed by taking advantage of the observation that deamidation of RhoA Gln 63 leads to decreased electrophoretic mobility on denaturing polyacrylamide gels. A time course of deamidation showed that wild type and L794P (the isoform used for structural determination) are equally active. As reported, substitution of Cys 866 by Ser completely abolishes activity. By comparison, Ala substitution of Asn 835 greatly reduces the catalytic rate but does not abolish activity, and Ala substitution of Ser 864 results in a small but distinguishable decrease in catalytic rate. Mutations of conserved residues on the face of CNF1 surrounding the active site pocket were designed to identify potential interactions with Rho. However, Ala substitution of Glu 943 or Asn 966, or Met substitution of Leu 769 fail to show any effect on activity.

Further, the present invention extends to any Rho GTPase activator that is capable of deamidating or transglutaminating Gln63 in RhoA and/or Gln61 in Rac1 or CDC42. Provided that the activator has such activity, then it may have as little as 50% sequence homology with CNF1 in the active site. Preferably, the activator shares absolute sequence homology where CNF1, CNF2 and DNT have sequence homology as shown in accompanying FIG. 5, although the present invention extends to such sequences lacking homology by 1, 2, 3, 4, or 5 amino acid residues. More preferably, the active site has at least 60% homology with CNF1. In this respect, the active site relates to residues 720 to 1010. More preferably, the active site of an activator of the present invention shares at least 70%, more preferably at least 90%, more preferably at least 95%, more preferably at least 99%, and most preferably at least 99.9% homology with CNF1 and CNF2 in the active site region bounded by residues 728 and 956.

The present invention also provides activated Rho-GTPases, for instance RhoA where Gln63 is deamidated or transglutaminated, and Rac1 or CDC42 where Gln61 is deamidated or transglutaminated.

Human RhoA is shown in SEQ ID NO. 4 (ras homolog gene family, member A), human Rac1 is shown in SEQ ID NO. 5 (ras-related C3 botulinum toxin substrate 1 isoform Rac1) and human CDC42 is shown in SEQ ID NO. 6 (cell division cycle 42 isoform 1). Of course, it will be appreciated that heterogeneity may result in small variations may occurring in these sequences throughout the population. Thus, where reference is made to RhoA, Rac1 and CDC42, it will be appreciated that this includes at least 70%, more preferably at least 90%, more preferably at least 95%, more preferably at least 99%, and most preferably at least 99.9% homology to the above-mentioned SEQ ID NOS, whilst retaining said amino acids at corresponding positions, as will be readily apparent to the skilled person. This also applies to activated Rho-GTPases of the present invention.

Chimeric molecules comprising a portion that has said homology attached to a second portion that has a further effector function, are also provided.

The activators of the present invention result in constitutive, therefore permanent, activation of Rho-GTPases. This is also particularly surprising.

In such a condition, the affected GTPase has reduced levels of hydrolytic activity, and is preferentially removed from the system by ubiquitinylation, and other processes, so that it is possible that the present invention may also be acting by a process of deletion of Rho GTPases in addition to, or instead of, activation of these enzymes [7].

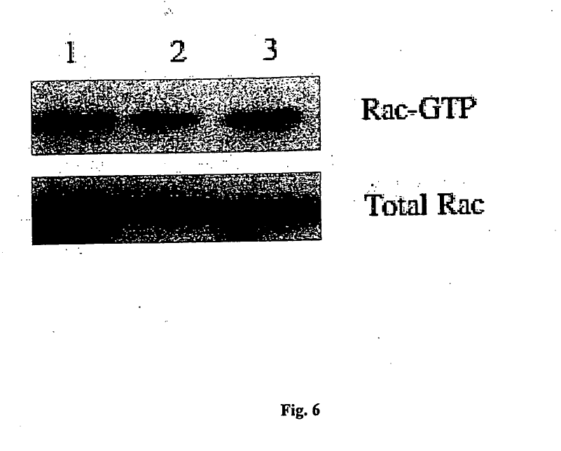

The effects of the activators of the present invention is a long term effect. For example, the mice used in the accompanying Examples were tested between one and four weeks after administration of low levels of toxin, and clearly demonstrated the learning advantages to be gained by administering activators of the present invention. The prolonged efficacy of CNF1 seems to parallel the time course of cerebral Rac activation, which is still observed 4 weeks post-injection (FIG. 6).

Activators of the present invention may be administered in any suitable form, but are preferably administered by injection. In mice, effects are seen in amounts as small as 0.6 fmol/kg. In such small quantities, it is generally preferably to target the dose and, as such, it is preferred to administer the activators of the present invention by, for example, lumbar puncture, intrathecally, or discrete injection into a selected area of the CNS, including the cerebral ventricles, as may be determined by the skilled physician.

General administration, such as p.o. (per oral), i.v. (intravenously), intramuscularly or transdermally is also possible, given the intrinsic ability of this class of proteins to cross plasma membranes. Given the sustained efficaciousness of the present invention, then such administrations need only be performed on no more than a weekly or monthly basis, and may be performed on an even more infrequent basis, such as twice yearly, or preferably on a 2- or 3-monthly basis. The molecules could be administered at any life time, including pre-natal or early postnatal, e.g. in order to prevent the effects of molecular deficits underlying the pathogenesis of inherited forms of mental retardation.

The activators of the present invention may be formulated in any suitable manner, such as in saline, and optionally with any buffering and/or isotonic agents. Quantities to be administered may be any that are readily determined by the skilled physician, taking into account such factors as age, weight and sex, but will generally vary between about 0.0001 fmol/kg and 1 μmol/kg, preferably between about 0.001 fmol/kg and 1 μmol/kg, and more preferably between about 0.01 and 100 fmol/kg.

The activators of the present invention may also be administered by gene therapy methods, such as delivery of a polynucleotide encoding the toxin linked to a suitable promoter via standard methods, such as encapsulation within a viral vector or delivery by “gene-gun” methods.

Thus, the present invention provides a polynucleotide, such as DNA or RNA, encoding the toxin or toxins, or mutants or variants thereof, preferably linked to a suitable promoter. The present invention also provides the use of said polynucleotides in therapy. Vectors, such as viral capsids, comprising or encompassing these polynucleotides are also provided.

The present invention further extends to nucleic acid sequences encoding activators of the present invention, to vectors comprising such sequences, whether they be DNA or RNA, to hosts comprising such sequences, and to methods of manufacture of activators of the invention comprising expressing all, part or a fusion protein comprising an activator from such hosts.

It will be appreciated that the present invention further extends to methods for the treatment of learning and cognitive disorders, said matters comprising administering an activator of the invention to a patient in need thereof.

The invention further provides a method for treating learning or cognitive disorders in a patient comprising administering a Rho GTPase activator selected from CNF1, CNF2, DNT or a mutant or variant thereof, provided that the Rho GTPase activator is effective either to deamidate or to transglutaminate Gln63 in RhoA and/or Gln61 in Rac1 and/or Gln61 in CDC42.

Conditions treatable by the activators of the present invention include any wherein the learning and/or cognitive processes are impaired, whether this be congenital or as a result of a condition developed later in life, for example. Thus, the invention is useful in the treatment and/or prophylaxis of the following conditions, for example: dementia associated with conditions such as Alzheimer's, and other types of dementia, including multi-infarctual, dementia associated with Parkinson's disease and Huntington's chorea and other, such as diffuse cerebral cortical atrophy, Lewy-body dementia, Pick's disease, mesolimbocortical dementia, and familial dementia with spastic paraparesis; and, Mild Cognitive Impairment and any other form of cognitive impairment associated with any condition, such as ADHD and schizophrenia, metabolic diseases, cerebro-vascular diseases, and psychic depression; mental retardation of any type, either genetic or induced by environmental factors. Neurodegenerative and lesional nervous system disorders will also directly benefit from the widespread effect of the treatment on the cytoskeleton and the consequent beneficial effect on the volume of the nervous tissue and its connectivity. These conditions include Amyotrophic Lateral Sclerosis, Parkinson's disease, cerebrovascular diseases, traumatic disorders of the central nervous system, Multiple Sclerosis, retinal degeneration. The activators of the present invention may further be used in increasing cognitive performances in healthy subjects.

In general, the present invention may be of assistance in other conditions where it is determined that the individual suffers from a reduced ability to learn from its surroundings.

### EXAMPLES

Rearrangement of dendritic spines in the CNS is thought to be associated with learning, and the Rho GTPase family controls actin dynamics, a mechanism whereby dendritic spine morphology is regulated. Thus, we decided to establish whether intracerebral injection of Cytotoxic Necrotising Factor 1 (CNF1), a toxin that contributes to Escherichia coli pathogenicity by sustained activation of RhoA, Rac1 and CDC42, could affect learning and memory in young adult CD1 and C57b16 mice. The molecule was injected once (20 icy, right hemisphere) at least 7 days before the start of the experiments.

### Methods

### Animals

The experiments were carried out on 2-month old male mice of two different strains: outbred albino CD1 and inbred C57b16 (Harlan Italy, S. Pietro at Natisone, UD, Italy). All procedures were carried out in accordance with the guidelines of the Council of European Communities 86/609/EEC and the protocols were approved by the Bioethical Committee of the Istituto Superiore di Sanità (Roma, Italy). All animals were housed under 12-h periods of light and darkness and constant temperature (20±2° C.) and humidity (55±5%) conditions. Food (Mucedola S.r.l., Settimo Milanese, Italy) and water were provided ad libitum.

### Bacterial Toxin Production and Administration

CNF1 was purified as previously described [9] from Escherichia coli strain 392 ISS (kindly provided by V. Falbo, ISS, Rome, Italy).

After general anaesthesia (Equithesin, 3 ml/Kg i.p.), the mice were mounted in a Krieg stereotaxic instrument (Stoelting, Chicago Ill., U.S.A.). The skin was incised in order to make the bregma visible. A 27 G needle attached to a 50 μl Hamilton microsyringe was pushed through the bone of the skull and positioned in the lateral ventricle of the right cerebral hemisphere. One minute after penetration, 2 μl of the test solution were injected. The experiment was performed in mice that had received a) 0.6 fmol/kg CNF1; b) 6.0 fmol/kg CNF1; c) saline; d) 0.6 fmol/kg CNF1 C866S, a recombinant toxin in which the change of cystein with serine at position 866 confers all the CNF1 properties except for the enzymatic activity on Rho GTPases [29]. The recombinant toxin CNF1 C866S was employed to demonstrate that the observed responses in experimental animals were due specifically to the ability of CNF1 to activate the Rho GTPases. Five minutes after injection, the needle was removed, and the surgical wound was sutured. The mice were then housed individually and monitored for one day. Experiments started at least one week post surgery.

### Fear Conditioning

Fear conditioning was carried out in transparent test chambers with electrified grid floor (ENV-008-FPU, Med Associates Inc., St. Albans, Vt., U.S.A.). In order to measure the degree of conditioning, two independent observers scored mice for immobility times (“freezing”) every 2 seconds. Conditioned stimulus (CS) was a pure tone (20 s duration, 4000 Hz, 85 dB), immediately followed by a continuously scrambled electric shock delivered in the grid floor (unconditioned stimulus, US: 2s, 0.75 mA, obtained with a Med Associates shocker-scrambler ENV-414S). Each tone-shock pairing was followed by a 64 s time during which immediate freezing was scored. After a baseline time (192 s) the mice received 5 tone-shock pairings. Twentyfour hours after the conditioning, the mice were placed back in the test chamber for 5 min and scored for freezing (contextual conditioning). Subsequently, they were moved to a novel chamber in which they were scored for freezing during a 192 s baseline time followed by a 320 s tone identical to the CS (cued conditioning). The mice were re-tested for cued conditioning 7 days later.

### Water Maze

### Apparatus

The behavioural test was performed in a silent room at a temperature of 24±1° C. The experimenter and the devices for data acquisition and analysis were located in an adjacent room.

Water maze is a circular pool of 80 cm diameter, 31 cm height, arbitrarily divided in 4 quadrants named according to the cardinal points (NE; NR; SE; SW) and filled with water made opaque with milk, at room temperature, up to a height of 21 cm.

In one quadrant, at the centre of the line from the pool wall to the pool centre, an 8-cm diameter, 20-cm high, water-filled Plexiglas cylinder was placed. The cylinder's upper surface, which had been roughened to facilitate climbing, was located 1 cm under the water and provided a platform on which the mouse could climb to escape from the water during the experiments. Under these conditions, the platform was invisible to the animals.

During the experiments, the operator was in the adjacent room and measured the escape latencies using a stopwatch. A camera mounted perpendicularly over the pool's centre captured mouse behaviour. All experiments were recorded on a PC for subsequent analysis.

### Behavioural Procedure.

The platform was held in a fixed position during the whole place learning. The mice were trained to learn the position in daily blocks of 3 consecutive trials. Altogether, the mice underwent 15 learning trials over 5 consecutive days. The following groups were studied: vehicle and CNF10.6 fmol/kg. In experiments carried out on C57b16 mice the effects of 0.6 fmol/kg CNF1 C866S were also studied. At the beginning of each trial, the animals were placed in water with their heads facing the pool wall, in the middle of one of the four wall segments. The starting point varied across trials according to a pseudo random sequence that was identical for all the mice. The mice were left in water until they reached the invisible platform and climbed on it; then they were left on the platform for a 10 s reinforcement period. If the platform had not been found within 70 s (cut-off time), the experimenter placed them on it.

Three days after the place learning the platform was removed and the mice were allowed to swim (spatial probe). The time spent in each pool quadrant during the first 30 s of swimming was determined, for subsequent analysis.

On the following day the platform was moved to a different quadrant and the mice were trained to finding it in 4 consecutive trials (reversal test, performed only on CD1 mice).

On a subsequent day, the animals were trained to find a platform that had been made visible with a sharply contrasted cylinder (5 cm diameter, 12 cm height) placed on it. The training consisted of 4 trials (60 min inter-trial interval). During this “cued learning”, the platform position and the starting point were changed every trial. This experiment was performed to test the animal's visual acuity, motor ability and motivation to locate the'platform.

### Hippocampal Slice Preparation and Electrophysiology

A separate group of CD1 mice treated with 0.6 fmol/kg CNF1 was used for in vitro electrophysiology. Mice were deeply anaesthetised with urethane (1.5 g/kg i.p.) and decapitated. The brains were removed and the hippocampus was isolated. Transverse hippocampal slices, 400 μm thick, were cut with a tissue chopper (The Mickle Laboratory Engineering Co. Ltd., Gomshall, Surrey, England), transferred to an incubation glass chamber containing artificial cerebrospinal fluid (AC SF) saturated with a gas mixture of 95% O₂ and 5% CO₂ and maintained at room temperature for at least 2 h. ACSF is a water solution (pH 7.4) containing (mM): 126 NaCl, 3.5 KCl, 1.2 NaH₂PO₄, 25 NaHCO₃, 2 CaCl₂, 1.3 MgCl₂, 11 glucose. For electrophysiological experiments, slices were transferred in a submerged-type recording chamber and perfused with oxygenated ACSF (24±1° C.) by a peristaltic pump (Gilson Minipulse 3) at a constant flow rate (2.5-3 ml/min). An electrode (stainless steel, 250 μm diameter, tapered tip size 8°, 5 MΩ; A-M Systems Inc., Carlsborg, Wash., USA) was placed into the stratum radiatum within the CA1 area to stimulate the Schaffer collateral-commissural fibres. Glass micropipettes (OD 1.0 mm, ID 0.7 mm, 1.5-2 MΩ) filled with ACSF were placed in the hippocampal cell body layer of the CA1 area for extracellular recording of Population Spike (PS) amplitudes, and in the hippocampal dendritic layer of the CA1 area for recording of field excitatory postsynaptic potentials (fEPSPs). The depth of the two electrodes was adjusted in order to maximise the height of the potentials, which were evoked by regular stimulation (0.033 Hz; squared waves, 100 μs; constant current). The responses were amplified 1000 times and filtered at 5 kHz (L-C low pass filter, 40 dB/decade). The signals were then sampled at 20 kHz, digitised (A/D board NB MIO 16 by National Instruments on personal computer Apple Macintosh IIfx) and stored on disk for subsequent off-line analysis.

Before the induction of LTP, basal neurotransmission was studied, recording input-output curves, i.e. the responses produced by 11 consecutive stimuli of linearly increasing intensity (0 to 200 μA in steps of 20 μA). The stimulus intensity used throughout the LTP experiments was 60 μA. For experimental purposes, only slices that had shown a steady response for at least 30 minutes were used. LTP was induced by three trains of tetanic stimuli (100 pulses, 100 Hz, 30 s inter-train interval, basal intensity) and recorded for 1 h, at least. Data were entered into analysis as a single subject, and therefore reflect individual mice.

Pull-down assay. Pull-down assay was performed as previously described [34]. Briefly, brains were homogenized in 50 mM TRIS (pH 7.4), 1 mM EDTA (pH 8.0), 0.5% NP40, 150 mM NaCl, 10% glycerol, 10 μg/ml aprotinin, 10 μg/ml leupeptin, 1 mM PMSF. The cleared homogenates were incubated with 50 μg of GST-PAK-CD fusion proteins bound to glutathione-coupled Sepharose beads (Amersham) for 40 min at 4° C. Beads were washed three times in the lysis buffer and bound proteins were eluted in sample buffer, subjected to SDS-PAGE and immunoblotted as already described⁵⁰. Whole-cell lysates were analyzed in parallel.

Fluorescence microscopy. Brains were frozen and cryosections of control and treated samples were obtained. Following flotation with 3.7% formaldehyde in PBS for 30 min at room temperature, sections were incubated with fluorescein isothiocyanate (FITC)-phalloidin (Sigma, dilution 1:300) at 37° C. for 30 min. After washing, sections were mounted with glycerol-phosphate-buffered saline and analyzed with an Olympus BX51 fluorescence microscope.

### Statistical Analysis

Data from fear conditioning were analysed by analysis of variance (ANOVA). Repeated measurement ANOVA was used to analyse the water maze data (one way on between-group comparison; two ways on repetition for place learning, one way on repetition for reversal and cued learning). T-test with Bonferroni's corrections was used for post hoc individual comparisons.

In electrophysiological experiments, input-output curves were analysed by repeated measurement ANOVA. LTP experiments were analysed by repeated measurement analysis of covariance (ANCOVA), using the average response values during the 5 min before tetanus as covariate. Owing to the high number of repeated measurements, in LTP experiments we used two levels of repetition, the lower of which grouped 4 values, i.e., 2 min intervals. The calculations were performed with Statistica 5.0 for Windows.

### Results

### General Observation

No animal deaths were observed during the experiments. Mice treated with the toxin did not exhibit any overt physical or behavioural abnormalities. No body weight or food and water intake analysis was carried out during the experimental time.

### Fear Conditioning

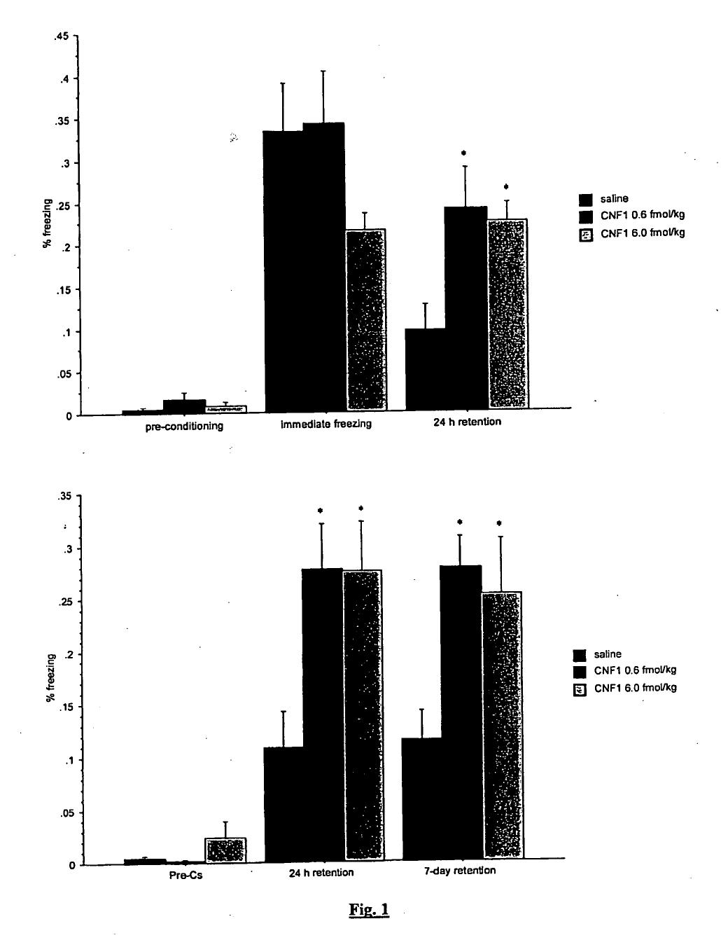

The results of fear conditioning are summarised in FIG. 1, which shows the enhancement of both context and cued conditioning in CNF1-treated CD1 mice. In the Figure, the percent of freezing time in mice injected icy with 0.6 fmol/kg CNF1 (n=12), 6.0 fmol/kg (n=12 for context test, n=11 for cued text) and saline (n=12 for context test, n=11 for cued text) 21-22 days before the conditioning is plotted. Data are expressed as mean±S.E.M.*p<0.05, significantly different from saline-treated group by t-test with Bonferroni's correction. On the whole, the data confirm the reduced tendency to freeze reported elsewhere for CD1 mice [1].

During the baseline of fear conditioning immobility was rather infrequent. Moreover, no significant difference was observed among the 3 groups (F_{2,33}=1.098, p=0.3456). The results of immediate freezing suggest that mice treated with 6.0 fmol/kg CNF1 are less subjected to freezing than those in the two other groups, although statistical analysis does not corroborate the result (F_{2,33}=1.986, p=0.1533).

Both context and cued conditioning demonstrated significant effects of CNF1 at 24 h post-conditioning. The analysis on freezing scores in the context test exhibited an increased freezing in treated animals, indicating an improvement in context learning induced by the treatment (F_{2,33}=4.848, p=0.0143; both 0.6 and 6.0 fmol/kg CNF1 significantly different from the vehicle). The significance was even higher if the freezing score recorded during conditioning was used as covariate (data not shown). Since context dependent conditioning is dependent on both the hippocampus and the amygdala, we examined data from cued conditioning, which is hippocampus independent. Twenty-four hours after conditioning, cued conditioning resulted in significant differences among groups (F_{2,31}=5.200, p=0.0113). Mice treated with both 0.6 and 6.0 CNF1 exhibited an increased response according to Bonferroni's test. The results obtained 7 days post conditioning confirmed the increases in freezing in the CNF1 treated groups (F_{2,31}=5.314, p=0.0104; both 0.6 and 6.0 fmol/kg CNF1 significantly different from the vehicle by Bonferroni's test); indicating that the effects of the toxin extend to long-term memory. The improved efficiency of both forms of conditioning suggests an overall enhancement of associative learning in treated mice. This could be explained by several factors, including a possible pain sensitising effect of CNF1. To test this hypothesis, we examined the mice for nociceptive threshold in the conditioning cages. The minimal amount of current required to elicit jumping and/or vocalising was determined. The average threshold currents were 330+34, 267+21, and 270+31 μA for mice treated with vehicle (n=5), CNF1 0.6 fmol/kg (n=12) and CNF1 6.0 fmol/kg (n=10). The differences were not significant by ANOVA (F_{2,24}=1.132; p=0.3390). Thus, increased sensitivity to pain induced by CNF1 cannot explain the increased freezing observed in both context and cued learning tests.

### Water Maze

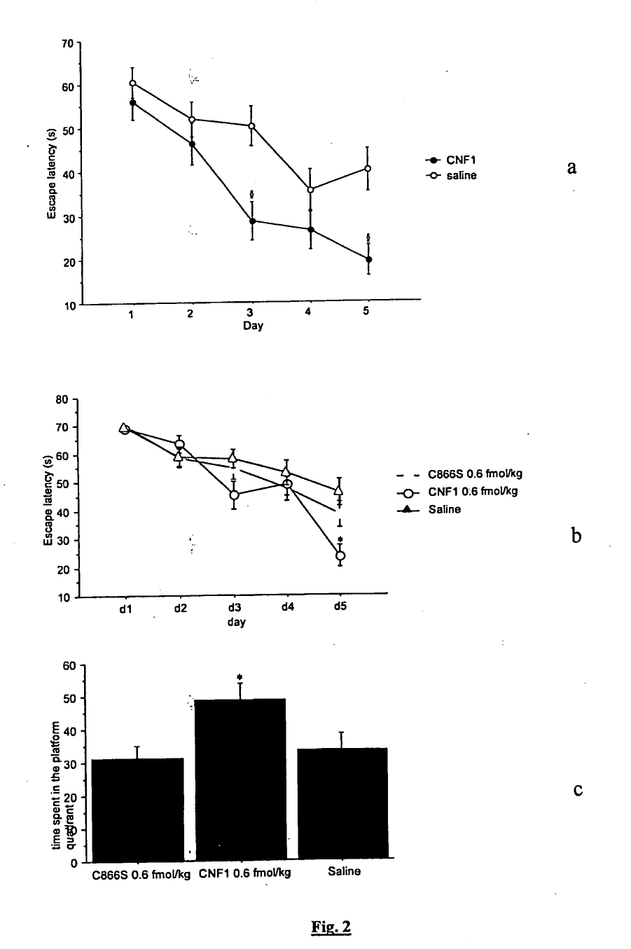

The results of the place learning are illustrated in FIG. 2, which shows the improved water-maze performances in CNF1-treated mice. FIG. 2a shows a summary of place learning performances in the water maze for CD1 mice. Data are mean±S.E.M. of escape latencies to reach the hidden platform (§ significantly different from saline-Mated, p<0.005). Mice were treated 10 days before the training with saline (n=11) or CNF1 0.6 fmol/kg (n=10).

The latency to escape to the platform decreased across the training sessions in both CNF1-treated and saline-treated mice (F_{4,76}=16.021, P<0.0001 by ANOVA for repeated measurements). The effect of the treatment appears to be significant (F_{1,19}=5.426, P=0.031). This indicates that learning occurs at different rates in the two groups. In particular, the CNF1-treated group performed better on day 3 (F_{1,19}=8.837, P=0.0078) and day 5 (F_{1,19}=5.584, P=0.0289), whereas no significant differences were observed in any of the other three intra-day comparisons.

FIG. 2b illustrates improved water maze performances in CNF1-treated C57b16 mice. Data are expressed as mean±S.E.M of escape latencies. Mice were injected i.c.v. with saline (n=12), 0.6 fmol/kg CNF1 (n=12), or 0.6 fmol/kg recombinant CNF1 (C866S, n=13) 10 days before the training. The ANOVA on data from the 5 days of training indicates significant differences in the rate of learning among groups (interaction “treatment” X “day of learning” F8,136=2.082, p=0.0416 by the ANOVA for repeated measurements). In particular, CNF1-treated mice performed better in the last day of training (*, P<0.05, significantly different from saline-treated group in the last day of training by ANOVA for repeated measurements and t-test with Bonferroni's correction).

The results of spatial probe (FIG. 2c) demonstrate a significant difference among groups of C57b16 mice in the time spent in the platform quadrant (0.6 fmol/kg CNF1: n=12; saline: n=12; C866S: n=13; mean percent time±S.E.M.; F2,34 4.145, p=0.0245;* significantly different from saline-treated group, p<0.05 by t-test with Bonferroni's correction). This again indicates that CNF1 improved spatial learning and that its effects can not be explained by learning through non-spatial strategies.

The ANOVA results for the reversal test in CD1 mice demonstrate that the difference among the average performance in the three groups just approaches statistical significance (F_{1,19}=3.391, P=0.0812). However, individual comparisons in trial 2 and trial 4 (t=2.746, DF=19, P=0.0129; t=2.115, DF=19, P=0.0478, respectively) show a significant enhancing effect of the treatment (data not shown).

The analysis of cued learning fails to demonstrate any significant difference among treatments and their interactions.

### Hippocampal Slice Electrophysiology

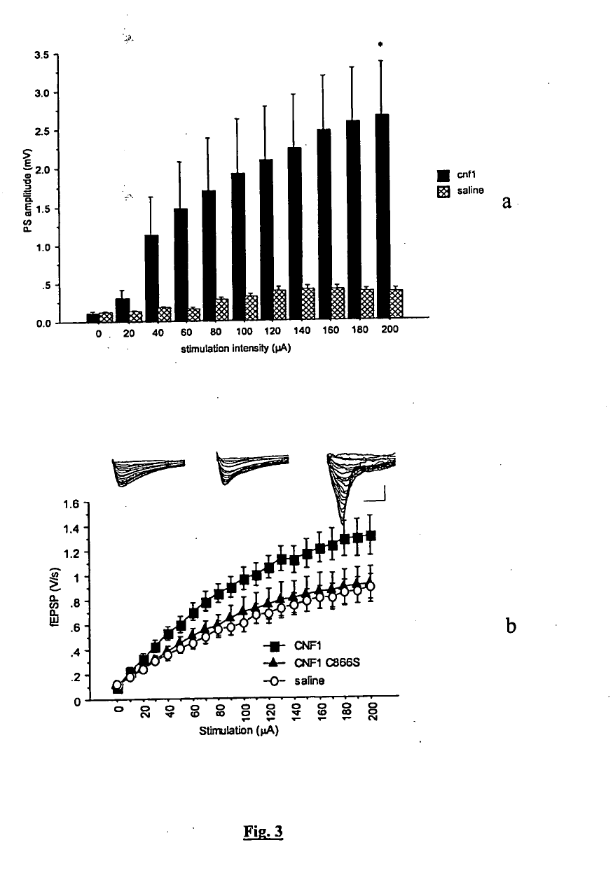

ANOVA analysis for repeated measurements indicated that the baseline responses recorded during the generation of input-output curves are significantly different in saline-treated CD1 mice as compared to mice treated with CNF1 0.6 fmol/kg. The results are illustrated in FIG. 3, showing the effects of CNF1 on hippocampal CA1 input-output curves. CNF1 (2 0.6 fmol/kg) and saline were injected icy 8-12 days before the recordings. Evoked responses were elicited by stimulation of Schaffer's collateral-commissural fibres (square waves, 100 ms, constant current) and recorded in the cell body layer. The responses are displayed as a function of stimulation intensity (μA). Each plot is the mean of the data obtained from 10 mice/treatment. Error bars: ±1 S.E.M.* significantly different from saline-treated, p<0.05.

PS amplitudes (FIG. 3a) were significantly affected both by the treatment (F_{1,18}=6.726, p=3.0183) and by stimulus intensity (F_{10,180}=12.658, p<0.0001). The interaction “treatment”*“intensity” was also significant (F_{10,180}=7.821, p<0.0001) demonstrating that the size of the differences depended on the stimulation intensity. After Bonferroni's correction, individual comparisons at the different intensity stimulation levels demonstrate a significant difference among the two treatments at 200 μA.

Input-output function of fEPSP slopes at CA3-CA1 synapse (FIG. 3a; means±S.E.M; saline, n=11; CNF1, n=10; CNF1 C866S, n=11) with representative traces (left, saline; center CNF1 C866S; right, CNF1; horizontal bar, 5 ms; vertical bar, 0.5 mV) confirm the enhancement of basal neurotransmission induced by CNF1 (treatment: F_{2,29}=3.600, P=0.0401, CNF1 significantly different from saline and CNF1 C866S; stimulation intensity: F_{20,580}=108.151, P<0.0001; treatment×intensity interaction: F_{40,580}=2.420, P<0.0001, by ANOVA for repeated measurements and post-hoc comparisons with Bonferroni's correction).

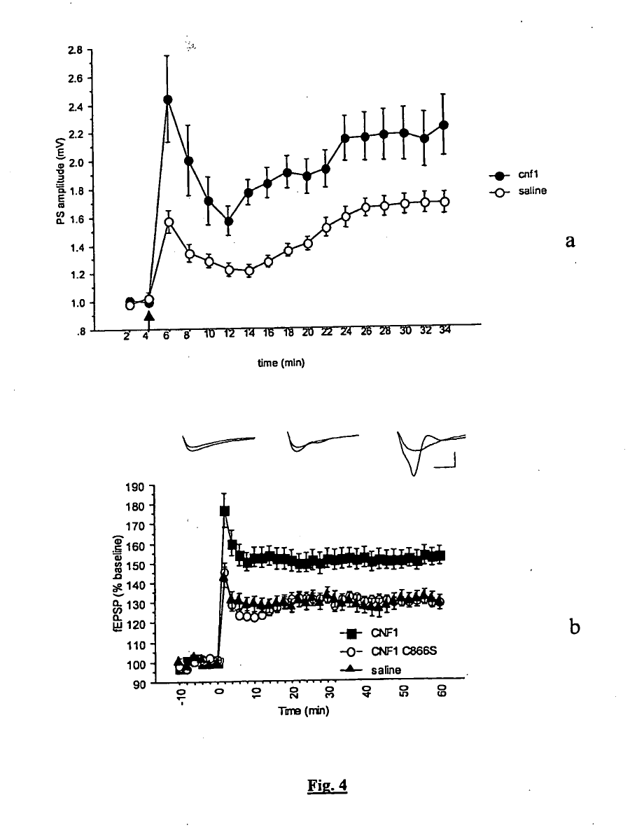

The results of ANCOVA on LTP of PS amplitude are illustrated in FIG. 4a, which shows the effects of CNF1 on CD1 mice. The normalised changes in the PS amplitude are displayed as a function of time. Each plot is the mean of the data obtained from mice injected with CNF1 (2 0.6 fmol/kg, n=10) and saline (2 n=10) 8-12 day before the recordings. Values were normalised with respect to the mean of baseline period 5 min prior to the delivery of tetanus. Tetanus (3 trains of 100 pulses at 100 Hz; 30 s inter-train interval; baseline pulse intensity and duration) was applied at the time indicated by the arrow. Error bars: ±1 S.E.M.

These results confirm the difference between the two treatments (F_{1,18}=12.415, p=0.0024). No significant differences emerged from the analysis of the interaction between the treatment and the main repetition factor, thus contradicting the possibility of different time trends of potentiation in the two groups.

The LTP of fEPSP slopes in CD1 mice (means±S.E.M.; saline, n=10; CNF1, n=8; CNF1 C866S, n=8) with representative traces (left, saline; center CNF1 C866S; right, CNF1; horizontal bar, 5 ms; vertical bar, 0.5 mV) is shown in FIG. 4b. The potentiation observed at 60 min post-tetanus was increased in the group treated with CNF1 (F_{2,22}=4.2502, P=0.0275 by ANCOVA), while the one observed in mice treated with the recombinant toxin matched the potentiation in the control group (F_{1,15}=0.0834, P=0.7767 by individual ANCOVA). The changes seem not to be caused by abnormal presynaptic function. In fact, a phenomenon sensitive to presynaptic changes such as paired-pulse facilitation (PPF), is not affected by either CNF1 or CNF1 C866S (treatment: F_{2,26}=0.3206, P=0.7285; treatment×interpulse interval interaction: F_{6,81}=0.2144, P=0.9712 by ANCOVA for repeated measurements; data not shown).

In a separate series of experiments, we explored the effect of 0.6 fmol/kg denatured CNF1 (100° C., 10 min) and 0.6 fmol/kg mutated CNF1 (i.e. cysteine in position 866 had been replaced with serine, a mutation that suppresses the GTPase activating property, [29]). In this set of experiments LTP and input-output curves matched those observed in saline-treated mice.

FIG. 6 illustrates that CNF1 causes persistent activation of Rac GTPase in brains of two-month old albino CD1 mice. Immunoblots, obtained by pull down experiments, show the amount of both total and activated Rac (Rac-GTP) in the left hippocampus at 4 weeks after single i.c.v. CNF1 had been injected in the right hemisphere (1) CNF1 6.0 fmol/kg; 2) CNF1 0.6 fmol/kg; 3) saline).

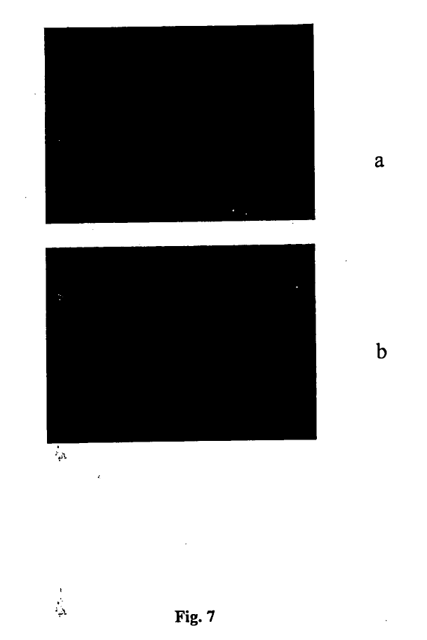

FIG. 7 shows that CNF1 enhances actin polymerization in the left parietal cortex of C57b16 mice. Mice were injected i.c.v. with saline or CNF1 in the right hemisphere 15 days before the experiments. Fluorescence micrographs of representative sections stained with FITC-phalloidin for F-actin detection are shown (magnification 40×). a) saline; b) 0.6 fmol/kg CNF1

### DISCUSSION

In a fear conditioning paradigm, 6 and 0.6 fmol/kg CNF1 increased both context- and cued-dependent freezing. The toxin also improved water maze performances, hippocampal CA1 glutamatergic neurotransmission and Long-Term Potentiation, whereas the same dose of mutated or denatured toxin was ineffective. The results suggest that pharmacological manipulation of Rho GTPases affect associative learning.

CNF1 has been shown to improve learning and memory in young CD1 and C57b16 mice, in the above Examples. The data from fear conditioning indicate an increased performance both in cued and in context-dependent learning, suggesting a general improvement of associative learning extending beyond hippocampal functioning. The increased performances in the cued test do not change the meaning of the result. Indeed, genetic enhancement of learning and memory, such as the one induced by manipulation of NMDA receptors [31], was associated with an increase of both context and tone conditioning. The finding is particularly significant when the fact that saline-treated mice displayed an increased freezing during the training (immediate freezing), is taken into account, which rules out an increased sensitivity to shock/fear in CNF1-treated mice. Differences in water maze performances confirm the general enhancement in learning abilities.

Increased hippocampal neurotransmission observed in input/output curve results might have played a role in the improved performances. It is received wisdom that hippocampus is essential for the temporary encoding of new information before being consolidated elsewhere [3]. LTP, which models the activity-dependent changes in synaptic efficacy that are believed to underlie learning, is enhanced in the 0.6 fmol/kg treated group and independently from the increased basal hippocampal neurotransmission. Rho signaling is intimately linked to actin filaments and cellular morphology and, consistently, we found that polymerized actin was overexpressed throughout the brain. One possible mechanism underlying the increased neurotransmission and improved learning might be represented by the increase in activated actin, as shown in FIG. 7. This action also represent a key mechanism for the trophic effect of Rho-GTPase activating bacterial toxins.

Most data concerning Rho GTPases have been obtained in peripheral tissue. We do not have a satisfactory knowledge of the actions of these proteins in the CNS. Moreover, regional differences in the Rho GTPase actions are likely to occur in different brain regions and neuronal types. These regional differences have not been satisfactorily studied yet. In addition, the biology of Rho GTPases may be different in the CNS as compared to the periphery. It has been independently shown that transfection of neurons from rat cerebral cortex, so that they encoded for constitutively activated Rac1 and CDC42, led to an increase in the number of dendrites per neuron, whereas dominant negative or inhibited forms of the proteins led to the opposite effect [33]. In the light of these results, it is conceivable that Rho family GTPases play a key role during the development of the CNS. However, neuronal morphogenesis occurs in adulthood as well, and it is likely to be dependent on the activity of this protein family.

Specific patterns of Rho-GTPase subtypes in the brain might still exist and be associated with selective central effects of CNF1. This may go some way toward explaining the inconsistency between the cognitive enhancement induced by CNF1, which is known to activate RhoA in periphery [6], and the reported effect of oligophrenia, which indirectly promotes de-activation of the same GTPase [4]. A possible correlation may also exist in the enhanced elimination of constitutively activated Rho GTPases [7].

As all experiments were performed between 1 and 4 weeks after a single toxin injection, the prolonged effect of CNF1 on behavioural and electrophysiology parameters was demonstrated. This sustained effect parallels the one observed in the activation of cerebral Rac following CNF1 injection: 28 days post-injection the amount of Rac-GTP is still increased as compared to control (FIG. 6). This is consistent with the apparent mechanism of action of the toxin, a permanent activation of Rho family GTPases [11].

### REFERENCES

[1] Adams, B., Fitch, T., Chaney, S, and Gerlai, R., Altered performance characteristics in cognitive tasks: comparison of the albino ICR and CD1 mouse strains, Behav Brain Res, 133 (2002) 351-61.
[2] Allen, K. M., Gleeson, J. G., Bagrodia, S., Partington, M. W., MacMillan, J. C., Cerione, R. A., Mulley, J. C. and Walsh, C. A., PAK3 mutation in nonsyndromic X-linked mental retardation, Nat Genet, 20 (1998) 25-30.
[3] Anagnostaras, S. G., Maren, S, and Fanselow, M. S., Temporally graded retrograde amnesia of contextual fear after hippocampal damage in rats: within-subjects examination, J Neurosci, 19 (1999) 1106-14.
[4] Billuart, P., Bienvenu, T., Ronce, N., des Portes, V., Vinet, M. C., Zemni, R., Roest Crollius, H., Carrie, A., Fauchereau, F., Chemy, M., Briault, S., Hamel, B., Fryns, J. P., Beldjord, C., Kahn, A., Moraine, C. and Chelly, J., Oligophrenin-1 encodes a rhoGAP protein involved in X-linked mental retardation, Nature, 392 (1998) 923-6.
[5] Buetow, L., Flatau, G., Chiu, K., Boquet, P. and Ghosh, P., Structure of the Rho-activating domain of Escherichia coli cytotoxic necrotizing factor 1, Nat Struct Biol, 8 (2001) 584-8.
[6] Buetow, L. and Ghosh, P., Structural elements required for deamidation of RhoA by cytotoxic necrotizing factor 1, Biochemistry, 42 (2003) 12784-91.
[7] Doye, A., Mettouchi, A., Bossis, G., Clement, R., Buisson-Touati, C., Flatau, G., Gagnoux, L., Piechaczyk, M., Boquet, P. and Lemichez, E., CNF1 exploits the ubiquitin-proteasome machinery to restrict Rho GTPase activation for bacterial host cell invasion, Cell, 111 (2002) 553-64.
[8] Edwards, D. C., Sanders, L. C., Bokoch, G. M. and Gill, G. N., Activation of LIM-kinase by Pak1 couples Rac/Cdc42 GTPase signalling to actin cytoskeletal dynamics, Nat Cell Biol, 1 (1999) 253-9.
[9] Falzano, L., Fiorentini, C., Donelli, G., Michel, E., Kocks, C., Cossart, P., Cabanie, L., Oswald, E. and Boquet, P., Induction of phagocytic behaviour in human epithelial cells by Escherichia coli cytotoxic necrotizing factor type 1, Mol Microbiol, 9 (1993) 1247-54.
[10] Fischer, M., Kaech, S., Knutti, D. and Matus, A., Rapid actin-based plasticity in dendritic spines, Neuron, 20 (1998) 847-54.
[11] Flatau, G., Lemichez, E., Gauthier, M., Chardin, P., Paris, S., Fiorentini, C. and Boquet, P., Toxin-induced activation of the G protein p21 Rho by deamidation of glutamine, Nature, 387 (1997) 729-33.
[12] Fukazawa, Y., Saitoh, Y., Ozawa; F., Ohta, Y., Mizuno, K. and Inokuchi, K., Hippocampal LTP is accompanied by enhanced F-actin content within the dendritic spine that is essential for late LTP maintenance in vivo, Neuron, 38 (2003) 447-60.
[13] Hall, A., G proteins and small GTPases: distant relatives keep in touch, Science, 280 (1998) 2074-5.
[14] Kimura, K., Ito, M., Amano, M., Chihara, K., Fukata, Y., Nakafuku, M., Yamamori, B., Feng, J., Nakano, T., Okawa, K., Iwamatsu, A. and Kaibuchi, K., Regulation of myosin phosphatase by Rho and Rho-associated kinase (Rho-kinase), Science, 273 (1996) 245-8.
[15] Kutsche, K., Yntema, H., Brandt, A., Jantke, I., Nothwang, H. G., Orth, U., Boavida, M. G., David, D., Chelly, J., Fryns, J. P., Moraine, C., Ropers, H. H., Hamel, B. C., van Bokhoven, H. and Gal, A., Mutations in ARHGEF6, encoding a guanine nucleotide exchange factor for Rho GTPases, in patients with X-linked mental retardation, Nat Genet, 26 (2000) 247-50.
[16] Lang, C., Barco, A., Zablow, L., Kandel, E. R., Siegelbaum, S. A. and Zakharenko, S. S., Transient expansion of synaptically connected dendritic spines upon induction of hippocampal long-term potentiation, Proc Natl Acad Sci USA, 101 (2004) 16665-70.
[17] Lendvai, B., Stern, E. A., Chen, B. and Svoboda, K., Experience-dependent plasticity of dendritic spines in the developing rat barrel cortex in vivo, Nature, 404 (2000) 876-81.
[18] Luo, L., Rho GTPases in neuronal morphogenesis, Nat Rev Neurosci, 1 (2000) 173-80.
[19] Luo, L., Hensch, T. K., Ackerman, L., Barbel, S., Jan, L. Y. and Jan, Y. N., Differential effects of the Rac GTPase on Purkinje cell axons and dendritic trunks and spines, Nature, 379 (1996) 837-40.
[20] Maekawa, M., Ishizaki, T., Boku, S., Watanabe, N., Fujita, A., Iwamatsu, A., Obinata, T., Ohashi, K., Mizuno, K. and Narumiya, S., Signaling from Rho to the actin cytoskeleton through protein kinases ROCK and LIM-kinase, Science, 285 (1999) 895-8.
[21] Muller, D., Toni, N. and Buchs, P. A., Spine changes associated with long-term potentiation, Hippocampus, 10 (2000) 596-604.
[22] Nakayama, A. Y., Harms, M. B. and Luo, L., Small GTPases Rac and Rho in the maintenance of dendritic spines and branches in hippocampal pyramidal neurons, J Neurosci, 20 (2000) 5329-38.
[23] O'Kane, E. M., Stone, T. W. and Morris, B. J., Activation of Rho GTPases by synaptic transmission in the hippocampus, J Neurochem, 87 (2003) 1309-12.
[24] O'Kane, E. M., Stone, T. W. and Morris, B. J., Increased long-term potentiation in the CA1 region of rat hippocampus via modulation of GTPase signalling or inhibition of Rho kinase, Neuropharmacology, 46 (2004) 879-87.
[25] Pasteris, N. G., Cadle, A., Logie, L. J., Porteous, M. E., Schwartz, C. E., Stevenson, R. E., Glover, T. W., Wilroy, R. S, and Gorski, J. L., Isolation and characterization of the faciogenital dysplasia (Aarskog-Scott syndrome) gene: a putative Rho/Rac guanine nucleotide exchange factor, Cell, 79 (1994) 669-78.
[26] Popov, V. I., Davies, H. A., Rogachevsky, V. V., Patrushev, I. V., Errington, M. L., Gabbott, P. L., Bliss, T. V. and Stewart, M. G., Remodelling of synaptic morphology but unchanged synaptic density during late phase long-term potentiation (LTP): a serial section electron micrograph study in the dentate gyrus in the anaesthetised rat, Neuroscience, 128 (2004) 251-62.
[27] Ramakers, G. J., Rho proteins, mental retardation and the cellular basis of cognition, Trends Neurosci, 25 (2002) 191-9.
[28] Rusakov, D. A., Davies, H. A., Harrison, E., Diana, G., Richter-Levin, G., Bliss, T. V. and Stewart, M. G., Ultrastructural synaptic correlates of spatial learning in rat hippocampus, Neuroscience, 80 (1997) 69-77.
[29] Schmidt, G., Selzer, J., Lerm, M. and Aktories, K., The Rho-deamidating cytotoxic necrotizing factor 1 from Escherichia coli possesses transglutaminase activity. Cysteine 866 and histidine 881 are essential for enzyme activity, J Biol Chem, 273 (1998) 13669-74.
[30] Takenawa, T. and Mild, H., WASP and WAVE family proteins: key molecules for rapid rearrangement of cortical actin filaments and cell movement, J Cell Sci, 114 (2001) 1801-9.
[31] Tang, Y. P., Shimizu, E., Dube, G. R., Rampon, C., Kerchner, G. A., Zhuo, M., Liu, G. and Tsien, J. Z., Genetic enhancement of learning and memory in mice, Nature, 401 (1999) 63-9.
[32] Tashiro, A., Minden, A. and Yuste, R., Regulation of dendritic spine morphology by the rho family of small GTPases: antagonistic roles of Rac and Rho, Cereb Cortex, 10 (2000) 927-38.
[33] Threadgill, R., Bobb, K. and Ghosh, A., Regulation of dendritic growth and remodeling by Rho, Rac, and Cdc42, Neuron, 19 (1997) 625-34.
[34] Travaglione, S., Messina, G., Fabbri, A., Falzano, L., Giammarioli, A. M., Grossi, M., Rufini, S, and Fiorentini, C., Cytotoxic necrotizing factor 1 hinders skeletal muscle differentiation in vitro by perturbing the activation/deactivation balance of Rho GTPases, Cell Death Differ (2004).
[35] Zito, K., Knott, G., Shepherd, G. M., Shenolikar, S, and Svoboda, K., Induction of spine growth and synapse formation by regulation of the spine actin cytoskeleton, Neuron, 44 (2004) 321-34.
[36] Endris, V., Wogatzky, B., Leimer, U., Bartsch, D., Zatyka, M., Latif, F., Maher, E. R., Tariverdian, G., Kirsch, S., Karch, D. and Rappold, G. A., The novel Rho-GTPase activating gene MEGAP/srGAP3 has a putative role in severe mental retardation, Proc Natl Acad Sci USA, 99 (2002) 11754-9.
[37] Govek, E. E., Newey, S. E., Akerman, C. J., Cross, J. R., Van der Veken, L. and Van Aelst, L., The X-linked mental retardation protein oligophrenin-1 is required for dendritic spine morphogenesis, Nat Neurosci, 7 (2004) 364-72.

All references and documents cited herein are hereby incorporate by reference.

1. Use of a Rho GTPase activator in the manufacture of a medicament for the treatment of learning and cognitive disorders, wherein the Rho GTPase activator is selected from CNF1, CNF2, DNT or a mutant or variant thereof, provided that the Rho GTPase activator is effective either to deamidate or to transglutaminate Gln63 in RhoA and/or Gln61 in Rac1 and/or Gln61 in CDC42.

2. Use according to claim 1, wherein the Rho GTPase activator is CNF1 (SEQ ID NO. 1) or a mutant or variant thereof that shares at least 70% homology thereto.

3. Use according to claim 1, wherein the Rho GTPase activator is CNF2 (SEQ ID NO. 2) or a mutant or variant thereof that shares at least 70% homology thereto.

4. Use according to claim 1, wherein the Rho GTPase activator is DNT (SEQ ID NO. 3) or a mutant or variant thereof that shares at least 70% homology thereto.

5. Use according to any of claims 2-4, wherein the mutant or variant shares at least 90% homology to said SEQ ID NO.

6. A bacterial Rho GTPase activator that is capable of deamidating or transglutaminating Gln63 in RhoA (SEQ ID NO. 4) and/or Gln61 in Rac1 (SEQ ID NO. 5) or CDC42 (SEQ ID NO. 6), or homologues having at least 90% sequence homology thereto whilst retaining said amino acids.

7. An activated Rho-GTPase, selected from RhoA (SEQ ID NO. 4) where Gln63 is deamidated or transglutaminated, and Rac1 (SEQ ID NO. 5) and CDC42 (SEQ ID NO. 6) where Gln61 is deamidated or transglumated.

8. A chimaeric molecule comprising the active site of a Rho GTPase activator, selected from CNF1, CNF2, DNT or a mutant or variant thereof as defined in any of claims 1-5, and a further element comprising a binding and/or translocation unit which contains all or part of an antibody molecule, specific for and capable of binding at least one target molecule.

9. A chimaeric molecule comprising the active site of a Rho GTPase activator, selected from CNF1, CNF2, DNT or a mutant or variant thereof as defined in any of claims 1-5, and a further element comprising a binding and/or translocation unit which contains part or all of a binding molecule, specific for and capable of binding at least one target receptor.

10. A chimaeric molecule according to claim 8 or 9, wherein Rho-GTPase activator is CNF1 and the active site is comprised within the catalytic domain of the activator, corresponding to 721-1013 of SEQ ID NO. 1.

11. A chimaeric molecule according to claim 8 or 9, wherein Rho-GTPase activator is CNF2 and the active site is comprised within the catalytic domain of the activator, corresponding to 721-1013 of SEQ ID NO. 2.

12. A chimaeric molecule according to claim 8 or 9, wherein Rho-GTPase activator is DNT and the active site is comprised within the catalytic domain of the activator, corresponding to 1167-1464 of SEQ ID NO. 3.

13. A chimaeric molecule according to claim 8, 9 or 10, wherein Rho-GTPase activator is CNF1 and the active site corresponds to residues 728 and 956 of SEQ ID NO. 1.

14. A chimaeric molecule according to claim 8, 9 or 11, wherein Rho-GTPase activator is CNF2 and the active site corresponds to residues 728 and 956 of SEQ ID NO. 2.

15. A method for treating learning or cognitive disorders in a patient comprising administering a Rho GTPase activator selected from CNF1, CNF2, DNT or a mutant or variant thereof, provided that the Rho GTPase activator is effective either to deamidate or to transglutaminate Gln63 in RhoA and/or Gln61 in Rac1 and/or Gln61 in CDC42.

16. A method according to claim 15, wherein the activator is administered by lumbar puncture, intrathecally, or discrete injection into a selected area of the CNS, including the cerebral ventricles.

17. A method according to claim 15, wherein the activator is administered per orally, intravenously, intramuscularly or transdermally.

18. A method according to any of claims 15-17, wherein the activator is administered as a polynucleotide encoding the activator, operably linked to a promoter, within a viral vector or capsid.

19. A method according to any of claims 15-18, for the treatment of prophylaxis of dementia associated with Alzheimer's disease, and dementia associated with Parkinson's disease and Huntington's chorea, diffuse cerebral cortical atrophy, Lewy-body dementia, Pick's disease, mesolimbocortical dementia, and familial dementia with spastic paraparesis; Mild Cognitive Impairment, AMID and schizophrenia, metabolic diseases, cerebro-vascular diseases, and psychic depression; mental retardation of any type, either genetic or induced by environmental factors, Neurodegenerative and lesional nervous system disorders including Amyotrophic Lateral Sclerosis, Parkinson's disease, cerebrovascular diseases, traumatic disorders of the central nervous system, Multiple Sclerosis, retinal degeneration.

20. A method according to any of claims 15-18, for increasing cognitive performances in healthy subjects.


SEQUENCE LISTING (6 sequences)

SEQ ID NO: 1 - protein, 1014 aa, Escherichia coli
  misc (721)..(1013): Catalytic Domain
MGNQWQQKYLLEYNELVSNFPSPERVVSDYIKNCFKTDLPWFSRIDPDNAYFICFSQNRSNSRSYTGWDHLGKYKTEVLTLTQAALINIGYRFDVFDDANSSTGIYKTKSADVFNEENEEKMLPSEYLHFLQKCDFAGVYGKTLSDYWSKYYDKFKLLLKNYYISSALYLYKNGELDEREYNFSMNALNRSDNISLFFFDIYGYYSSDIFVAKNNDKVMLFIPGAKKPFLFKKNIADLRLTLKELIKDSDNKQLLSQHFSLYSRQDGVSYAGVNSVLHAIENDGNFNESYFLYSNKTLSNKDVFDAIAISVKKRSFSDGDIVIKSNSEAQRDYALTILQTILSMTPIFDIVVPEVSVPLGLGIITSSMGISFDQLINGDTYEERRSAIPGLATNAVLLGLSFAIPLLISKAGINQGVLSSVINNEGRTLNETNIDIFLKEYGIAEDSIPSTNVLDVKLKSSGQHVNIVKLSDEDNQIVAVKGSSLSGIYYEVDIETGYEILSRRIYRTEYNNEILWTRGGGLKGGQPFDFESLNIPVFFKDEPYSAVTGSPLSFINDDSSLLYPDTNPKLPQPTSEMDIVNYVKGSGSFGDRFVTLMRGATEEEAWNIASYHTAGGSTEELHEILLGQGPQSSLGFTEYTSNVNSADAASRRHFLVVIKVHVKYINNNNVSYVNHWAIPDEAPVEVLAVVDRRFNFPEPSTSPDISTIRKLLSLRYFKESIESTSKSNFQKLSRGNIDVLKGRGGISSTRQRAIYPYFEAANADEQQPLFFYIKKDRFDNHGYDQYFYDNTVGLNGIPTLNTYTGEIPSDSSSLGSTYWKKYNLTNETSIIRVSNSARGANGIKIALEEVQEGKPVIITSGNLSGCTTIVARKEGYIYKVHTGTTKSLAGFTSTTGVKKAVEVLELLTKEPIPRVEGIMSNDFLVDYLSENFEDSLITYSSSEKKPDSQITIIRDNVSVFPYFLDNIPEHGFGTSATVLVRVDGNVVVRSLSESYSLNADVSEISVLKVFSKKF

SEQ ID NO: 2 - protein, 1014 aa, Escherichia coli
  misc (721)..(1013): Catalytic Domain
MNVQWQQKYLLEYNELVSNFPSPERVVSDYIRRCFKTDLPWFSQVDPDNTYFIRFSQSRSNSRSYTGWDHLGKYKTGVLTLTQAALINIGYHFDVFDDANASAGIYKTSSADMFNEKNEEKMLPSEYLYFLKGCDFSGIYGRFLSDYWSKYYDKFKLLLKNYYISSALYLYKNGEIDEYEYNFSISALNRRDNISLFFFDIYGYYSSDMFVAKNNERVMLFIPGAKKPFLFEKNIADLRISLKNLIKENDNKQLLSQHFSLYSRQDGITYAGVNSVLNAIENDGVFNESYFLYSNKRINNKDVFDAVAFSVKKRSFSDGDIVIKSNSEAQRDYALTILQTILSMTPIFDVAIPEVSVTLGLGIIASSMGISFDQLINGDTYEERRSAIPGLATNAALLGLSFAIPFLISKAGTNQKILSRYTKHEIRTLNETNIDMFLEEYGINKNSISETKVLEVELKGSGQHVNIVKLSDEDNKIVAVKGNSLSGIYYEVDIETGYEISSRRIYRTEYNDKIFWTRGGGLKGGQSFDFESLKLPIFFKDEPYSAVPGSSLSFINDDSSLLYPNSTPKLPQPTPEMEIVNYVKRAGNFGERLVTLMRGTTEEEAWNIARYHTAGGSTEELHEILLGQGPQSSLGFTEYTSNINSADAASRRHFLVVIKVQVKYINNNNVSHVNHWAIPDEAPVEVLAVVDRRFNFPEPSTPPNISIIHKLLSLRYFKENIESTSRLNLQKLNRGNIDIFKGRGSISSTRQRAIYPYFESANADEQQPVFFYIKKNRFDDFGYDQYFYNSTVGLNGIPTLNTYTGEILSDASSLGSTYWKKYNLTNETSIIRVSNSARGANGIKIALEEVQEGKPVIITSGNLSGCTTIVARKGGYLYKVHTGTTIPLAGFTSTTGVKKAVEVFELLTNNPMPRVEGVMNNDFLVNYLAESFDESLITYSSSEQKIGSKITISRDNVSTFPYFLDNIPEKGFGTSVTILVRVDGNVIVKSLSESYSLNVENSNISVLHVFSKDF

SEQ ID NO: 3 - protein, 1464 aa, Bordetella pertussis
  misc (1167)..(1464): Catalytic domain
MDKDESALRQLVDMALVGYDGVVEELLALPSEESGDLAGGRAKREKAEFALFSEAPNGDEPIGQDARTWFYFPKYRPVAVSNLKKMQVAIRARLEPESLILQWLIALDVYLGVLIAALSRTVISDLVFEYVKARYEIYYLLNRVPHPLATAYLKRRRQRPVDRSGRLGSVFEHPLWFAYDELAGTVDLDADIYEQALAESIERRMDGEPDDGSLDTAEHDVWRLCRDGINRGEQAIFQASGPYGVVADAGYMRTVADLAYADALADCLHAQLRIRAQGSVDSPGDEMPRKLDAWEIAKFHLAATQQARVDLLEAAFALDYAALRDVRVYGDYRNALALRFIKREALRLLGARRGNASTMPAVAAGEYDEIVASGAANDAAYVSMAAALIAGVLCDLESAQRTLPVVLARFRPLGVLARFRRLEQETAGMLLGDQEPEPRGFISFTDFRDSDAFASYAEYAAQFNDYIDQYSILEAQRLARILALGSRMTVDQWCLPLQKVRHYKVLTSQPGLIARGIENHNRGIEYCLGRPPLTDLPGLFTMFQLHDSSWLLVSNINGELWSDVLANAEVMQNPTLAALAEPQGRFRTGRRTGGWFLGGPATEGPSLRDNYLLKLRQSNPGLDVKKCWYFGYRQEYRLPAGALGVPLFAVSVALRHSLDDLAAHAKSALYKPSEWQKFAFWIVPFYREIFFSTQDRSYRVDVGSIVFDSISLLASVFSIGGKLGSFTRTQYGNLRNFVVRQRIAGLSGQRLWRSVLKELPALIGASGLRLSRSLLVDLYEIFEPVPIRRLVAGFVSATTVGGRNQAFLRQAFSAASSSAGRTGGQLASEWRMAGVDATGLVESTSGGRFEGIYTRGLGPLSECTEHFIVESGNAYRVIWDAYTHGWRVVNGRLPPRLTYTVPVRLNGQGHWETHLDVPGRGGAPEIFGRIRTRNLVALAAEQAAPMRRLLNQARRVALRHIDTCRSRLALPRAESDMDAAIRIFFGEPDAGLRQRIGRRLQEVRAYIGDLSPVNDVLYRAGYDLDDVATLFNAVDRNTSLGRQARMELYLDAIVDLHARLGYENARFVDLMAFHLLSLGHAATASEVVEAVSPRLLGNVFDISNVAQLERGIGNPASTGLFVMLGAYSESSPAIFQSFVNDIFPAWRQASGGGPLVWNFGPAAISPTRLDYANTDIGLLNHGDISPLRARPPLGGRRDIDLPPGLDISFVRYDRPVRMSAPRALDASVFRPVDGPVHGYIQSWTGAEIEYAYGAPAAAREVMLTDNVRIISIENGDEGAIGVRVRLDTVPVATPLILTGGSLSGCTTMVGVKEGYLAFYHTGKSTELGDWATAREGVQALYQAHLAMGYAPISIPAPMRNDDLVSIAATYDRAVIAYLGKDVPGGGSTRITRHDEGAGSVVSFDYNAAVQASAVPRLGQVYVLISNDGQGARAVLLAEDLAWAGSGSALDVLNERLVTLFPAPV

SEQ ID NO: 4 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 5 - protein, 192 aa, Homo sapiens
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 6 - protein, 192 aa, Homo sapiens; identical to SEQ ID NO: 5